(19) United States
(12) Patent Application Publication
(10) Pub. No.: US 2021/0290590 A1
(43) Pub. Date: Sep. 23, 2021

(54) METHODS OF TREATING SUBJECTS INFECTED WITH HIV

(71) Applicant: Janssen Sciences Ireland Unlimited Company, Co Cork (IE)

(72) Inventors: David ANDERSON, New Hope, PA (US); Wing CHOW, Belle Mead, NJ (US); Keith J. DUNN, Philadelphia, PA (US); Donghan LUO, Randolph, NJ (US); Richard E. NETTLES, New Hope, PA (US); Richard Bruce SIMONSON, Southampton, PA (US)

(21) Appl. No.: 17/199,831

(22) Filed: Mar. 12, 2021

Related U.S. Application Data
(60) Provisional application No. 62/989,328, filed on Mar. 13, 2020.

(51) Int. Cl. A61K 31/34; A61K 9/00; A61K 31/5377; A61K 31/513; A61K 31/675; A61P 31/18

(52) U.S. Cl. CPC A61K 31/34; A61K 9/0053; A61P 31/18; A61K 31/513; A61K 31/675; A61K 31/5377

(57) ABSTRACT

The disclosure is directed to methods of treating humans diagnosed with an HIV infection and who experience weight gain while being treated with an integrase inhibitor regimen, as well as a novel HIV inhibitor regime for use in the treatment of those humans.

### CROSS-REFERENCE TO RELATED APPLICATION

This application claims the benefit of U.S. Provisional Application No. 62/989,328, filed Mar. 13, 2020, the entirety of which is incorporated by reference herein.

### TECHNICAL FIELD

The disclosure is directed to methods of treating humans diagnosed with an HIV infection and who experience weight gain while being treated with an integrase inhibitor regimen, as well as a novel HIV inhibitor regime for use in the treatment of those humans.

### BACKGROUND

Increases in body weight and/or BMI are of clinical concern, as obesity can lead to other metabolic complications such as hypertension, changes in lipid parameters, and/or insulin resistance, all of which ultimately may increase cardiovascular risk. Antiviral regimens that prevent or slow the rate of body weight gain in HIV-infected subjects are needed.

### SUMMARY

The disclosure is directed to methods of treating humans infected with an HIV virus and exhibiting an HIV viral load of less than or equal to 50 copies of HIV-1 virus particles per mL of blood plasma (<50c/mL), comprising orally administering to the human, once daily, a protease inhibitor regimen comprising: darunavir, or a hydrate or solvate thereof; cobicistat; emtricitabine; and tenofovir alafenamide, or a pharmaceutically acceptable salt thereof; wherein the human is treatment experienced and is switched to the protease inhibitor regimen from a first anti-retroviral regimen comprising: an integrase inhibitor, tenofovir alafenamide, or a pharmaceutically acceptable salt thereof, and emtricitabine; and wherein the human has experienced a ≥10% increase in weight within a 12-month time period during the administration of the first anti-retroviral regimen; and wherein the subject exhibits a viral load of less than or equal to 50 copies of HIV-1 virus particles per mL of blood plasma (<50c/mL) after at least 24 weeks of the once-daily administration of the protease inhibitor regimen. The disclosure is also directed to the above protease inhibitor regime for use the treatment of the above defined patient population.

### DETAILED DESCRIPTION OF ILLUSTRATIVE EMBODIMENTS

The present disclosure may be understood more readily by reference to the following detailed description taken in connection with the accompanying examples, which form a part of this disclosure. It is to be understood that this invention is not limited to the specific devices, methods, applications, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated compounds, which allows the presence of only the named compounds, along with any pharmaceutically carriers, and excludes other compounds.

All ranges disclosed herein are inclusive of the recited endpoint and independently combinable (for example, the range of “from 2 mg to 10 mg” is inclusive of the endpoints, 2 mg and 10 mg, and all the intermediate values). The endpoints of the ranges and any values disclosed herein are not limited to the precise range or value; they are sufficiently imprecise to include values approximating these ranges and/or values.

As used herein, approximating language may be applied to modify any quantitative representation that may vary without resulting in a change in the basic function to which it is related. Accordingly, a value modified by a term or terms, such as “about” and “substantially,” may not be limited to the precise value specified, in some cases. In at least some instances, the approximating language may correspond to the precision of an instrument for measuring the value. The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9 to 1.1. Other meanings of “about” may be apparent from the context, such as rounding off, so, for example “about 1” may also mean from 0.5 to 1.4.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound of the disclosure that is pharmaceutically acceptable and that possesses the desired pharmacological activity of the parent compound. In particular, such salts are non-toxic and may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like. See, e.g., U.S. Food & Drug Administration, Pharmaceutical Quality/CMC Guidances.

“Pharmaceutically acceptable excipient” refers to a diluent, adjuvant, excipient or carrier with which a compound of the disclosure is administered. A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of excipients include calcium carbonate, calcium phosphate, various sugars and types of starch, cellulose derivatives, gelatin, vegetable oils, stearates, silicon dioxide, polyvinyl alcohols, talc, titanium dioxide, ferric oxide, and polyethylene glycols. See, e.g., U.S. Food & Drug Administration, Pharmaceutical Quality/CMC Guidances.

“Subject” includes humans. The terms “human,” “patient,” and “subject” can be used interchangeably herein. Humans treated according to the methods of the disclosure may be adults, that is, ages 18 years or older. In other aspects, human treated according to the methods of the disclosure may be pediatric humans, that is, less than 18 years of age. In some aspects, the pediatric patients treated according to the disclosed methods weight at least 40 kg.

“Treating” or “treatment” of any disease or disorder refers, in one embodiment, to ameliorating the disease or disorder (i.e., arresting or reducing the development of the disease or at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder. As used herein, treating a human infected with an HIV virus refers to the administration of an anti-retroviral regimen to the subject with the goal of maintaining, regaining, or initiating HIV suppression, e.g., HIV viral load of <50 copies/mL blood plasma.

“Single unit dosage form” as used herein refers to dosage forms suitable for oral administration such as, but not limited to, tablets, capsules, gelcaps, and caplets.

Methods of diagnosing an HIV infection are known in the art and include ELISA tests, home tests, saliva tests, viral load tests, and Western Blots. An exemplary assay is Roche COBAS AmpliPrep/COBAS TaqMan HIV-1 Test, Version 2.0.

The present disclosure is directed to methods of treating a human infected with an HIV virus, in particular an HIV-1 virus, who is virologically suppressed, i.e., HIV RNA (e.g., HIV-1 RNA)<50 copies/mL (c/mL) blood plasma. The humans treated according to the described methods will have been treated with a first antiviral regimen that includes an integrase inhibitor, for example, dolutegravir, elvitegravir, elvitegravir/cobicistat, or bictegravir. In preferred aspects, the first antiviral regimen includes an integrase inhibitor, a pharmaceutically acceptable salt of tenofovir alafenamide (e.g., tenofovir alafenamide fumarate), and emtricitabine. For clarity, a human who has been administered a “first antiviral regimen” as used herein refers to a human who has been administered an antiviral regimen prior to the protease inhibitor regimen described herein and may include a human that has been administered one or more antiviral regimens in combination or sequentially prior to the protease inhibitor regimen described herein. Accordingly, in certain aspects, a human that is treated with a first anti-retroviral regimen for a period of time (e.g., a 6-month to 12-month period of time or longer) may refer to a human previously treated with one or more anti-retroviral regimens prior to said period of time. In certain aspects, a human that is treated with a first anti-retroviral regimen for a period of time (e.g., a 6-month to 12-month period of time or longer) has not been previously treated with another anti-retroviral regimen prior to the first anti-retroviral regimen prior to said period of time. The humans treated according to the described methods, during the course of being treated with the first anti-retroviral regimen, will have experienced a rapid weight gain within a 12-month time period during the course of the first anti-retroviral regimen. “Rapid weight gain,” as used herein, refers to an increase in weight of 10% or more in a 12-month time period. In some aspects, rapid weight gain refers to an increase in weight of 10% or more in a 6-month time period. Rapid weight gain can be accompanied by other clinically relevant effects, for example, decreases in body composition as measured by dual-energy X-ray absorptiometry (DEXA), increases in waist circumference, increases in blood pressure, e.g., systolic and/or diastolic blood pressure, increases in fasting lipid, e.g., triglycerides and/or total cholesterol, increases in fasting glucose, increases in homeostatic model assessment of insulin resistance (HOMA-IR), increases in HbA1c, or a combination thereof.

In accordance with the disclosed methods, virologically suppressed humans who have experienced rapid weight gain while being treated with an integrase inhibitor anti-retroviral regimen (a first anti-retroviral regimen) are switched to a protease inhibitor regimen. As used herein, the switch to a protease inhibitor regimen refers to the human stopping administration of the integrase inhibitor regimen (the first anti-retroviral regimen) and starting administration of a protease inhibitor regimen. According to the disclosure, the protease inhibitor regimen to which the human is switched excludes an integrase inhibitor and comprises:
darunavir, or a hydrate or solvate thereof;
cobicistat;
emtricitabine; and
tenofovir alafenamide, or a pharmaceutically acceptable salt thereof.

Significantly, when the described methods are performed, the human will remain virologically suppressed after the switch to the protease inhibitor regimen from the first anti-retroviral regimen that included an integrase inhibitor. In addition, whereas the human will have experienced rapid weight gain during administration of the first anti-retroviral regimen, as well as optionally experiencing concomitant, clinically relevant effects associated with rapid weight gain, after switching to the protease inhibitor regimen, the human will not exhibit a clinically significant change in body weight, e.g., will not exhibit a clinically significant increase in body weight, after about 24 weeks or after about 48 weeks of the administration of the protease inhibitor regimen.

In some aspects, after switching to the administration of the protease inhibitor regimen, the human will experience a clinically significant change, e.g. a clinically significant decrease, in body weight, after about 24 weeks or after about 48 weeks of the administration of the protease inhibitor regimen.

In some aspects, after switching to administration of the protease inhibitor regimen, the human will experience clinically significant changes, e.g., will exhibit clinically significant improvements, in, for example, body composition as measured by dual-energy X-ray absorptiometry (DEXA), waist circumference, blood pressure, e.g., systolic and/or diastolic blood pressure, fasting lipids, e.g., triglycerides and/or total cholesterol, fasting glucose, homeostatic model assessment of insulin resistance (HOMA-IR), and HbA1c, or improvements in a combination thereof. In certain aspects, improvements in body composition include improvements in the mass and/or volume of trunk Visceral Adipose Tissue (VAT), total body fat, adjusted total body fat (total body minus head), and appendage fat (legs and arms). In certain aspects, improvements in body composition include improvements in the relative amount of fat in a selected region (e.g. trunk, total body, adjusted total body, or appendages).

In some aspects of the methods of the disclosure, the human does not exhibit a clinically significant change in body weight, as compared to the human's body weight prior to administering the protease inhibitor regimen, after about 24 weeks, e.g., after 24 weeks. In some aspects of the disclosure, the human does not exhibit a clinically significant change in body weight, as compared to the human's body weight prior to administering the protease inhibitor regimen, after about 48 weeks, e.g., after 48 weeks. As used herein, “no clinically significant change in body weight” refers to a body weight change of less than 5%, for example, a 0, 1, 1.5, 2, 2.5, 3, 3.5, 4, or 4.5% change in body weight, as compared to baseline.

In some aspects of the disclosure, the human exhibits a clinically significant change in body weight, as compared to the human's body weight prior to administering the protease inhibitor regimen, after about 24 weeks, e.g., after 24 weeks. In some aspects of the disclosure, the human exhibits a clinically significant change in body weight, as compared to the human's body weight prior to administering the protease inhibitor regimen, after about 48 weeks, e.g., after 48 weeks. As used herein, a “clinically significant change in body weight” refers to a body weight change of at least 5%, for example, a 5, 5.5, 6, 6.5, 7, 7.5, 8, 8.5, 9, 9.5, or 10% change in body weight, as compared to baseline.

In some aspects of the disclosure, the human exhibits a clinically significant loss in body weight, as compared to the human's body weight prior to administering the protease inhibitor regimen, after about 24 weeks, e.g., after 24 weeks. In some aspects of the disclosure, the human exhibits a clinically significant loss in body weight, as compared to the human's body weight prior to administering the protease inhibitor regimen, after about 48 weeks, e.g., after 48 weeks. As used herein, a “clinically significant loss in body weight” refers to a body weight loss of at least 5%, for example, a 5, 5.5, 6, 6.5, 7, 7.5, 8, 8.5, 9, 9.5, or 10% loss in body weight, as compared to baseline.

In some aspects of the disclosure, the human exhibits no clinically significant change in body composition as measured by dual-energy X-ray absorptiometry (DEXA), as compared to the human's body composition as measured by DEXA prior to administering the protease inhibitor regimen, after about 24 weeks, e.g., after 24 weeks. In some aspects of the disclosure, the human exhibits no clinically significant change in body composition as measured by DEXA, as compared to the human's body composition as measured by DEXA prior to administering the protease inhibitor regimen, after about 48 weeks, e.g., after 48 weeks. As used herein, “no clinically significant change in body composition as measured by DEXA” refers to a change in T-score of 10% or less, for example, a 10, 9, 8, 7, 6, 5, 4, 3, 2, 1, or 0% change in T-score, as compared to baseline.

In some aspects of the disclosure, the human exhibits a clinically significant improvement in body composition as measured by DEXA, as compared to the human's body composition as measured by DEXA prior to administering the protease inhibitor regimen, after about 24 weeks, e.g., after 24 weeks. In some aspects of the disclosure, the human exhibits a clinically significant improvement in body composition as measured by DEXA, as compared to the human's body composition as measured by DEXA prior to administering the protease inhibitor regimen, after about 48 weeks, e.g., after 48 weeks. As used herein, “a clinically significant improvement in body composition as measured by DEXA” refers to an improvement in T-score of more than 10%, for example, a 10.5, 11, 11.5, 12, 12.5, 13, or 13.5% improvement in T-score, as compared to baseline.

In some aspects of the disclosure, the human exhibits no clinically significant change in total body fat as measured by DEXA, as compared to the human's total body fat as measured by DEXA prior to administering the protease inhibitor regimen, after about 24 weeks, e.g., after 24 weeks. In some aspects of the disclosure, the human exhibits no clinically significant change in total body fat as measured by DEXA, as compared to the human's total body fat as measured by DEXA prior to administering the protease inhibitor regimen, after about 48 weeks, e.g., after 48 weeks. As used herein, “no clinically significant change in total body fat as measured by DEXA” refers to a change in total body fat of 10% or less, for example, a 10, 9, 8, 7, 6, 5, 4, 3, 2, 1, or 0% change in total body fat, as compared to baseline.

In some aspects of the disclosure, the human exhibits a clinically significant improvement in total body fat as measured by DEXA, as compared to the human's total body fat as measured by DEXA prior to administering the protease inhibitor regimen, after about 24 weeks, e.g., after 24 weeks. In some aspects of the disclosure, the human exhibits a clinically significant improvement in total body fat as measured by DEXA, as compared to the human's total body fat as measured by DEXA prior to administering the protease inhibitor regimen, after about 48 weeks, e.g., after 48 weeks. As used herein, “a clinically significant improvement in total body fat as measured by DEXA” refers to a decrease in total body fat of more than 10%, for example, a 15, 20, 25, 30, 35, 40, 45, 50% or greater decrease in total body fat, as compared to baseline.

In some aspects of the disclosure, the human exhibits no clinically significant change in adjusted total body fat as measured by DEXA, as compared to the human's adjusted total body fat as measured by DEXA prior to administering the protease inhibitor regimen, after about 24 weeks, e.g., after 24 weeks. In some aspects of the disclosure, the human exhibits no clinically significant change in adjusted total body fat as measured by DEXA, as compared to the human's adjusted total body fat as measured by DEXA prior to administering the protease inhibitor regimen, after about 48 weeks, e.g., after 48 weeks. As used herein, “no clinically significant change in adjusted total body fat as measured by DEXA” refers to a change in adjusted total body fat of 10% or less, for example, a 10, 9, 8, 7, 6, 5, 4, 3, 2, 1, or 0% change in total body fat, as compared to baseline.

In some aspects of the disclosure, the human exhibits a clinically significant improvement in adjusted total body fat as measured by DEXA, as compared to the human's adjusted total body fat as measured by DEXA prior to administering the protease inhibitor regimen, after about 24 weeks, e.g., after 24 weeks. In some aspects of the disclosure, the human exhibits a clinically significant improvement in adjusted total body fat as measured by DEXA, as compared to the human's adjusted total body fat as measured by DEXA prior to administering the protease inhibitor regimen, after about 48 weeks, e.g., after 48 weeks. As used herein, “a clinically significant improvement in adjusted total body fat as measured by DEXA” refers to a decrease in adjusted total body fat of more than 10%, for example, a 15, 20, 25, 30, 35, 40, 45, 50% or greater decrease in total body fat, as compared to baseline.

In some aspects of the disclosure, the human exhibits no clinically significant change in appendage (legs and arms) fat as measured by DEXA, as compared to the human's appendage fat as measured by DEXA prior to administering the protease inhibitor regimen, after about 24 weeks, e.g., after 24 weeks. In some aspects of the disclosure, the human exhibits no clinically significant change in appendage fat as measured by DEXA, as compared to the human's appendage fat as measured by DEXA prior to administering the protease inhibitor regimen, after about 48 weeks, e.g., after 48 weeks. As used herein, “no clinically significant change in appendage fat as measured by DEXA” refers to a change in appendage fat of 10% or less, for example, a 10, 9, 8, 7, 6, 5, 4, 3, 2, 1, or 0% change in appendage fat, as compared to baseline.

In some aspects of the disclosure, the human exhibits a clinically significant improvement in appendage fat as measured by DEXA, as compared to the human's appendage fat as measured by DEXA prior to administering the protease inhibitor regimen, after about 24 weeks, e.g., after 24 weeks. In some aspects of the disclosure, the human exhibits a clinically significant improvement in appendage fat as measured by DEXA, as compared to the human's appendage fat as measured by DEXA prior to administering the protease inhibitor regimen, after about 48 weeks, e.g., after 48 weeks. As used herein, “a clinically significant improvement in appendage fat as measured by DEXA” refers to a decrease in appendage fat of more than 10%, for example, a 15, 20, 25, 30, 35, 40, 45, 50% or greater decrease in total body fat, as compared to baseline.

In some aspects of the disclosure, the human exhibits no clinically significant change in visceral adipose tissue (VAT) volume as measured by DEXA, as compared to the human's VAT volume as measured by DEXA prior to administering the protease inhibitor regimen, after about 24 weeks, e.g., after 24 weeks. In some aspects of the disclosure, the human exhibits no clinically significant change in VAT volume as measured by DEXA, as compared to the human's VAT volume as measured by DEXA prior to administering the protease inhibitor regimen, after about 48 weeks, e.g., after 48 weeks. As used herein, “no clinically significant change in VAT volume as measured by DEXA” refers to a change in VAT volume of 10% or less, for example, a 10, 9, 8, 7, 6, 5, 4, 3, 2, 1, or 0% change in VAT volume, as compared to baseline.

In some aspects of the disclosure, the human exhibits a clinically significant improvement in VAT volume as measured by DEXA, as compared to the human's VAT volume as measured by DEXA prior to administering the protease inhibitor regimen, after about 24 weeks, e.g., after 24 weeks. In some aspects of the disclosure, the human exhibits a clinically significant improvement in VAT volume as measured by DEXA, as compared to the human's VAT volume as measured by DEXA prior to administering the protease inhibitor regimen, after about 48 weeks, e.g., after 48 weeks. As used herein, “a clinically significant improvement in VAT volume as measured by DEXA” refers to a decrease in VAT volume of more than 10%, for example, a 15, 20, 25, 30, 35, 40, 45, 50% or greater decrease in VAT volume, as compared to baseline.

In some aspects of the disclosure, the human exhibits no clinically significant change in waist circumference, as compared to the human's waist circumference prior to administering the protease inhibitor regimen after about 24 weeks, e.g., after 24 weeks. In some aspects of the disclosure, the human exhibits no clinically significant change in waist circumference, as compared to the human's waist circumference prior to administering the protease inhibitor regimen after about 48 weeks, e.g., after 48 weeks. As used herein, “no clinically significant change in waist circumference” refers to a change in waist circumference of less than 5 cm, for example, 4.5, 4, 3.5, 3, 2.5, 2, 1.5, 1, 0.5, or 0 cm change in waist circumference, as compared to baseline.

In some aspects of the disclosure, the human exhibits a clinically significant improvement in waist circumference, as compared to the human's waist circumference prior to administering the protease inhibitor regimen after about 24 weeks, e.g., after 24 weeks. In some aspects of the disclosure, the human exhibits a clinically significant improvement in waist circumference, as compared to the human's waist circumference prior to administering the protease inhibitor regimen after about 48 weeks, e.g., after 48 weeks. As used herein, “a clinically significant improvement in waist circumference” refers to a reduction in waist circumference by at least 5 cm, for example, 5, 5.5, 6, 6.5, 7, 7.5, 8, 8.5, 9, 9.5, or 10 cm reduction in waist circumference, as compared to baseline. In certain aspects, the human is a male having a waist circumference greater than or equal to 40 inches prior to administering the protease inhibitor regimen and exhibits a waist circumference of less than 40 inches at about 48 weeks after administering the protease inhibitor regimen. In certain aspects, the human is a female having a waist circumference greater than or equal to 35 inches prior to administering the protease inhibitor regimen and exhibits a waist circumference of less than 35 inches at about 48 weeks after administering the protease inhibitor regimen.

In some aspects of the disclosure, the human exhibits no clinically significant change in blood pressure, as compared to the human's blood pressure prior to administering the protease inhibitor regimen after about 24 weeks, e.g., after 24 weeks. In some aspects of the disclosure, the human exhibits no clinically significant change in blood pressure, as compared to the human's blood pressure prior to administering the protease inhibitor regimen after about 48 weeks, e.g., after 48 weeks. As used herein, “no clinically significant change in blood pressure” refers to a change in systolic blood pressure of less than 10 mmHg, for example, 9.5, 9, 8.5, 8, 7.5, 7, 6.5, 6, 5.5, 5, 4.5, 4, 3.5, 3, 2.5, 2, 1.5, 1, 0.5, or 0 mmHg change in systolic blood pressure, as compared to baseline. Alternatively, “no clinically significant change in blood pressure” refers to a change in diastolic blood pressure of less than 5 mmHg, for example, 4.5, 4, 3.5, 3, 2.5, 2, 1.5, 1, 0.5, or 0 mmHg change in diastolic blood pressure, as compared to baseline. No clinically significant change in blood pressure can also refer to no clinically significant changes in both systolic and diastolic blood pressures.

In some aspects of the disclosure, the human exhibits a clinically significant improvement in blood pressure, as compared to the human's blood pressure prior to administering the protease inhibitor regimen after about 24 weeks, e.g., after 24 weeks. In some aspects of the disclosure, the human exhibits a clinically significant improvement in blood pressure, as compared to the human's blood pressure prior to administering the protease inhibitor regimen after about 48 weeks, e.g., after 48 weeks. As used herein, “a clinically significant improvement in blood pressure” refers to a decrease in systolic blood pressure of 10 mmHg or greater, for example, 10, 10.5, 11, 11.5, 12, 12.5, 13, 13.5, 14, 14.5, or 15 mmHg decrease in systolic blood pressure, as compared to baseline. Alternatively, “a clinically significant decrease in diastolic pressure” refers to a decrease in diastolic blood pressure of 5 mmHg or greater, for example, 5, 5.5, 6, 6.5, 7, 7.5, 8, 8.5, 9, 9.5, or 10 mmHg decrease in diastolic blood pressure, as compared to baseline. Clinically significant improvement in blood pressure can also refer to improvements in both systolic and diastolic blood pressures.

In some aspects of the disclosure, the human exhibits no clinically significant change in fasting lipids, as compared to the human's fasting lipids prior to administering the protease inhibitor regimen after about 24 weeks, e.g., after 24 weeks. In some aspects of the disclosure, the human exhibits no clinically significant change in fasting lipids, as compared to the human's fasting lipids prior to administering the protease inhibitor regimen after about 48 weeks, e.g., after 48 weeks. As used herein, “no clinically significant change in fasting lipids” refers to a change of 5 mg/dL or less, e.g., 5, 4, 3, 2, or 1 mg/dL, in fasting triglycerides, as compared to baseline. In other aspects, “no clinically significant change in fasting lipids” refers to a change of 5 mg/dL or less, e.g., 5, 4, 3, 2, or 1 mg/dL, in fasting total cholesterol, as compared to baseline.

In some aspects of the disclosure, the human exhibits a clinically significant improvement in fasting lipids, as compared to the human's fasting lipids prior to administering the protease inhibitor regimen after about 24 weeks, e.g., after 24 weeks. In some aspects of the disclosure, the human exhibits a clinically significant improvement in fasting lipids, as compared to the human's fasting lipids prior to administering the protease inhibitor regimen after about 48 weeks, e.g., after 48 weeks. As used herein, “a clinically significant improvement in fasting lipids” refers to a decrease of more than 5 mg/dL, e.g., 6, 7, 8, 9, 10, 11, 12, 13, 14, 15, 16, 17, 18, 19 or more mg/dL, in fasting triglycerides, as compared to baseline. In other aspects, “a clinically significant improvement in fasting lipids” refers to a decrease of more than 5 mg/dL, e.g., 6, 7, 8, 9, 10, 11, 12, 13, 14, 15, 16, 17, 18, 19 or more mg/dL, in fasting total cholesterol, as compared to baseline.

In some aspects of the disclosure, the human exhibits no clinically significant change in fasting glucose, as compared to the human's fasting glucose prior to administering the protease inhibitor regimen after about 24 weeks, e.g., after 24 weeks. In some aspects of the disclosure, the human exhibits no clinically significant change in fasting glucose, as compared to the human's fasting glucose prior to administering the protease inhibitor regimen after about 48 weeks, e.g., after 48 weeks. As used herein, “no clinically significant change in fasting glucose” refers to a change of 5 mg/dL or less, e.g., 5, 4, 3, 2, or 1 mg/dL, in fasting glucose, as compared to baseline.

In some aspects of the disclosure, the human exhibits a clinically significant improvement in fasting glucose, as compared to the human's fasting glucose prior to administering the protease inhibitor regimen after about 24 weeks, e.g., after 24 weeks. In some aspects of the disclosure, the human exhibits a clinically significant improvement in fasting glucose, as compared to the human's fasting glucose prior to administering the protease inhibitor regimen after about 48 weeks, e.g., after 48 weeks. As used herein, “a clinically significant improvement in fasting glucose” refers to a decrease of more than 5 mg/dL, e.g., 6, 7, 8, 9, 10, 11, 12, 13, 14, 15, 16, 17, 18, 19 or more mg/dL, in fasting glucose, as compared to baseline.

In some aspects, the human exhibits no clinically significant change in homeostatic model assessment of insulin resistance (HOMA-IR), as compared to the human's HOMA-IR prior to administering the protease inhibitor regimen after about 24 weeks, e.g., after 24 weeks. In some aspects, the human exhibits no clinically significant change in HOMA-IR, as compared to the human's HOMA-IR prior to administering the protease inhibitor regimen after about 48 weeks, e.g., after 48 weeks. As used herein, “no clinically significant change in HOMA-IR” refers to a change of less than 10%, e.g., 9, 8, 7, 6, 5, 4, 3, 2, 1, or less %, in HOMA-IR, as compared to baseline.

In some aspects, the human exhibits a clinically significant decrease in HOMA-IR, as compared to the human's HOMA-IR prior to administering the protease inhibitor regimen after about 24 weeks, e.g., after 24 weeks. In some aspects, the human exhibits a clinically significant decrease in HOMA-IR, as compared to the human's HOMA-IR prior to administering the protease inhibitor regimen after about 48 weeks, e.g., after 48 weeks. As used herein, “a clinically significant decrease in HOMA-IR” refers to a change of 10% or more, e.g., 10, 11, 12, 13, 14, 15, 16, 17, 18, 19 or more %, in HOMA-IR, as compared to baseline.

In some aspects, the human exhibits no clinically significant change in HbA1c, as compared to the human's HbA1c prior to administering the protease inhibitor regimen after about 24 weeks, e.g., after 24 weeks. In some aspects, the human exhibits no clinically significant change in HbA1c, as compared to the human's HbA1c prior to administering the protease inhibitor regimen after about 48 weeks, e.g., after 48 weeks. As used herein, “no clinically significant change in HbA1c” refers to a change of less than 10%, e.g., 9, 8, 7, 6, 5, 4, 3, 2, 1, or less %, in HbA1c, as compared to baseline.

In some aspects, the human exhibits a clinically significant decrease in HbA1c, as compared to the human's HbA1c prior to administering the protease inhibitor regimen after about 24 weeks, e.g., after 24 weeks. In some aspects, the human exhibits a clinically significant decrease in HbA1c, as compared to the human's HbA1c prior to administering the protease inhibitor regimen after about 48 weeks, e.g., after 48 weeks. As used herein, “a clinically significant decrease in HbA1c” refers to a change of 10% or more, e.g., 10, 11, 12, 13, 14, 15, 16, 17, 18, 19 or more %, in HbA1c, as compared to baseline.

In some aspects of the disclosure, the first antiviral regimen is administered to the human concurrently with an anti-hypertensive, anti-hyperglycemic, or a lipid lowering agent. In certain aspects, switching to the protease inhibitor regimen described herein permits a dose-reduction or complete withdrawal of the anti-hypertensive, anti-hyperglycemic, or lipid lowering agent (e.g. about 6, 12, 18, 24, 30, 36, 42, or 48 weeks following the switch to the protease inhibitor regimen).

According to the disclosure, the human may be administered a protease inhibitor regimen comprising darunavir. Darunavir ([(1S,2R)-3-[[(4-aminophenyl)sulfonyl](2-methylpropyl)amino]-2-hydroxy-1-(phenylmethyl)propyl]-carbamic acid (3R,3aS,6aR)-hexahydrofuro[2,3-b] furan-3-yl ester) is a human immunodeficiency virus protease inhibitor and has the following structure:

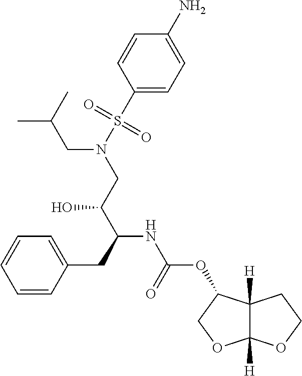 [SMILES: CC(C)CN(C[C@@H](O)[C@H](Cc1ccccc1)NC(=O)O[C@H]1CO[C@H]2OCC[C@H]21)S(=O)(=O)c1ccc(N)cc1]

In other aspects, the human in need of treatment may be administered a darunavir solvate, for example darunavir ethanolate (e.g., PREZISTA®), darunavir propylene glycolate, darunavir glycolate, darunavir propanolate, and the like. For example, in some aspects, the human in need of treatment is administered darunavir ethanolate. In other aspects, the human in need of treatment is administered darunavir propylene glycolate. In other aspects, the human in need of treatment is administered darunavir glycolate. In other aspects, the human in need of treatment is administered darunavir propanolate.

In some aspects, the human in need of treatment may be administered a darunavir hydrate.

According to the disclosure, the human in need is administered the darunavir, the darunavir solvate, or the darunavir hydrate in a therapeutically effective amount. Preferably, the human is administered the therapeutically effective amount, once daily. In other aspects, the human is administered the daily, therapeutically effective amount in divided doses, for example, in two doses per day or in three doses per day. The therapeutically effective amount can be determined by those skilled in the art, and will generally be in the amount equivalent to about 600 mg to about 1000 mg of darunavir, per day. In some aspects, the therapeutically effective amount of the darunavir, the darunavir solvate, or the darunavir hydrate will be an amount equivalent to about 600, 650, 700, 750, 800, 850, 900, 950, or 1000 mg of darunavir, per day. In some aspects, the human in need is administered about 800 mg of darunavir, once daily. For example, the therapeutically effective amount of the darunavir, the darunavir solvate, or the darunavir hydrate will be an amount equivalent to about 600, 650, 700, 750, 800, 850, 900, 950, or 1000 mg of darunavir, once daily.

In some aspects, the human in need is administered about 800 mg of darunavir, per day. In some aspects, the human in need is administered about 800 mg of darunavir, once daily.

In other aspects, the human in need is administered a darunavir solvate, in an amount equivalent to about 800 mg of darunavir, per day. In other aspects, the human in need is administered darunavir ethanolate, in an amount equivalent to about 800 mg of darunavir, per day. In other aspects, the human in need is administered darunavir ethanolate, in an amount equivalent to about 800 mg of darunavir, once daily. In other aspects, the human in need is administered darunavir propylene glycolate, in an amount equivalent to about 800 mg of darunavir, per day. In other aspects, the human in need is administered darunavir propylene glycolate, in an amount equivalent to about 800 mg of darunavir, once daily. In other aspects, the human in need is administered darunavir glycolate, in an amount equivalent to about 800 mg of darunavir, per day. In other aspects, the human in need is administered darunavir glycolate, in an amount equivalent to about 800 mg of darunavir, once daily. In other aspects, the human in need is administered darunavir propanolate, in an amount equivalent to about 800 mg of darunavir, per day. In other aspects, the human in need is administered darunavir propanolate, in an amount equivalent to about 800 mg of darunavir, once daily.

In other aspects, the human in need is administered a darunavir hydrate, in an amount equivalent to about 800 mg of darunavir, per day. In other aspects, the human in need is administered a darunavir hydrate, in an amount equivalent to about 800 mg of darunavir, once daily.

According to the disclosure, the human in need is also administered cobicistat (1,3-thiazol-5-ylmethyl [(2R,5R)-5-{[(2S)-2-[(methyl {[2-(propan-2-yl)-1,3-thiazol-4-yl]methyl}carbamoyl)amino]-4-(morpholin-4-yl)butanoyl]amino}-1,6-diphenylhexan-2-yl]carbamate), a CYP3A inhibitor. Cobicistat has the following structure:

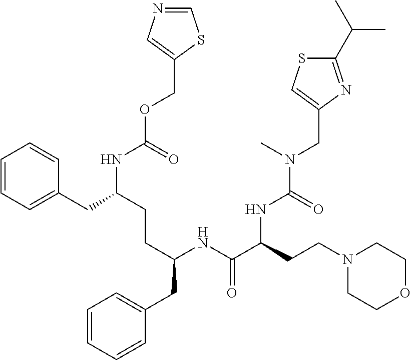 [SMILES: CC(C)c1nc(CN(C)C(=O)N[C@@H](CCN2CCOCC2)C(=O)N[C@H](CC[C@H](Cc2ccccc2)NC(=O)OCc2cncs2)Cc2ccccc2)cs1]

According to the disclosure, the human in need is administered the cobicistat in a therapeutically effective amount. Preferably, the human in need is administered the therapeutically effective amount, once daily. In other aspects, the human in need is administered the daily, therapeutically effective amount in divided doses, for example, in two doses per day or in three doses per day. The therapeutically effective amount can be determined by those skilled in the art, and will generally be in the amount equivalent to about 100 mg to about 200 mg of cobicistat, per day. For example, the therapeutically effective amount of cobicistat administered will be an amount equivalent to about 100, 125, 150, 175, or 200 mg of cobicistat, per day. For example, the therapeutically effective amount of cobicistat administered will be an amount equivalent to about 100, 125, 150, 175, or 200 mg of cobicistat, once daily. In some aspects, the human in need is administered about 150 mg of cobicistat, per day. In some aspects, the human in need is administered about 150 mg of cobicistat, once daily.

According to the disclosure, the human in need is also administered tenofovir alafenamide or a pharmaceutically acceptable salt thereof. Tenofovir alafenamide is a nucleotide analog reverse transcriptase inhibitor and has the following structure:

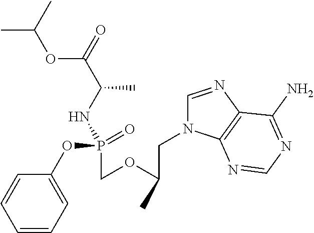

According to the disclosure, the human in need is administered the tenofovir alafenamide in a therapeutically effective amount. Preferably, the human in need is administered the therapeutically effective amount, once daily. In other aspects, the human in need is administered the daily, therapeutically effective amount in divided doses, for example, in two doses per day or in three doses per day. The therapeutically effective amount of tenofovir alafenamide will generally be in the amount equivalent to about 5 mg to about 300 mg of tenofovir, per day. In other aspects, the therapeutically effective amount of tenofovir alafenamide administered will generally be in the amount equivalent to about 5 mg to about 300 mg of tenofovir alafenamide, per day. For example, the therapeutically effective amount of tenofovir alafenamide administered will be an amount equivalent to about 5, 10, 15, 20, 25, 30, 35, 40, 45, 50, 55, 60, 65, 70, 75, 80, 85, 90, 95, 100, 125, 150, 175, 200, 225, 250, 275, or 300 mg of tenofovir alafenamide, per day. In some aspects, the therapeutically effective amount of tenofovir alafenamide administered will be an amount equivalent to about 5, 10, 15, 20, 25, 30, 35, 40, 45, 50, 55, 60, 65, 70, 75, 80, 85, 90, 95, 100, 125, 150, 175, 200, 225, 250, 275, or 300 mg of tenofovir alafenamide, once daily.

In some aspects, the human may be administered a pharmaceutically acceptable salt of tenofovir alafenamide. Preferably, the human is administered a therapeutically effective amount of the pharmaceutically acceptable salt of tenofovir alafenamide, once daily. In other aspects, the human is administered the daily, therapeutically effective amount of the pharmaceutically acceptable salt of tenofovir alafenamide in divided doses, for example, in two doses per day or in three doses per day.

For example, the human in need may be administered a pharmaceutically acceptable salt of tenofovir alafenamide, for example tenofovir alafenamide fumarate. When administering tenofovir alafenamide fumarate, a therapeutically effective amount will be an amount equivalent to about 10 mg to about 25 mg of tenofovir alafenamide, per day. In some aspects, the tenofovir alafenamide fumarate will be administered in an amount equivalent to 10, 11, 12, 13, 14, 15, 16, 17, 18, 19, 20, 21, 22, 23, 24, or about 25 mg of tenofovir alafenamide, per day. For example, the tenofovir alafenamide fumarate will be administered in an amount equivalent to 10, 11, 12, 13, 14, 15, 16, 17, 18, 19, 20, 21, 22, 23, 24, or about 25 mg of tenofovir alafenamide, once daily.

According to the disclosure, the human is also administered emtricitabine in a therapeutically effective amount. Emtricitabine is nucleoside reverse-transcriptase inhibitor and has the following structure:

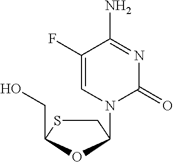 [SMILES: Nc1nc(=O)n([C@@H]2CS[C@H](CO)O2)cc1F]

Preferably, the human is administered the therapeutically effective amount of emtricitabine, once daily. In other aspects, the human is administered the daily, therapeutically effective amount in divided doses, for example, in two doses per day or in three doses per day. The therapeutically effective amount will generally be about 150 mg to about 200 mg of emtricitabine, per day. For example, the therapeutically effective amount of the emtricitabine will be about 150, 160, 170, 180, 190, or about 200 mg of emtricitabine, per day. In some aspects, the therapeutically effective amount of the emtricitabine will be about 150, 160, 170, 180, 190, or about 200 mg of emtricitabine, once daily.

In other exemplary embodiments, darunavir, a darunavir solvate, or a darunavir hydrate; cobicistat; tenofovir, tenofovir alafenamide or a pharmaceutically acceptable salt of tenofovir alafenamide; and emtricitabine can be combined together into a single unit dosage form. For example, a single unit dosage form of the disclosure may comprise a darunavir solvate; cobicistat, a pharmaceutically acceptable salt of tenofovir alafenamide; and emtricitabine. An exemplary single unit dosage form is available under the tradename SYMTUZA®, (darunavir ethanolate (equivalent to 800 mg of darunavir), cobicistat (150 mg) absorbed onto silicon dioxide, tenofovir alafenamide fumarate (equivalent to 10 mg of tenofovir alafenamide), and emtricitabine (200 mg)) and SYMTUZA® (darunavir ethanolate (equivalent to 800 mg of darunavir), cobicistat (150 mg) absorbed onto silicon dioxide, tenofovir alafenamide fumarate (equivalent to 10 mg of tenofovir alafenamide), and emtricitabine (200 mg)), among others.

In some aspects, the single unit dosage forms of the disclosure may be used in combination with additional active ingredients in the treatment of HIV infections. The additional active ingredients may be co-administered separately with a single unit dosage form of the disclosure. The combination may serve to increase efficacy (e.g., by including in the combination a compound potentiating the potency or effectiveness of an active agent according to the disclosure), decrease one or more side effects, or decrease the required dose of the active agent according to the disclosure.

Other products that can be used with the methods of the disclosure include, PREZCOBIX® (cobicistat (150 mg), darunavir (800 mg), PREZISTA® (suspension, darunavir 100 mg/mL, tablet 75 mg, 150 mg, 600 mg, or 800 mg), TYBOST® (cobicistat, 150 mg), EMTRIVA® (suspension, emtricitabine 10 mg/mL, capsule 200 mg), DESCOVY® (emtricitabine 200 mg, tenofovir alafenamide fumarate 25 mg base equivalent), TRUVADA® (emtricitabine, tenofovir disoproxil fumarate), VEMLIDY® (tenofovir alafenamide fumarate, 25 mg base equivalent), and VIREAD® (tenofovir disoproxil fumarate).

Oral dosage forms, for example, tablets, of the disclosure may include an active agent according to the disclosure mixed with pharmaceutically acceptable excipients such as inert diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, microcrystalline cellulose, silicified microcrystalline cellulose, mannitol, sorbitol, and the like. Starch, polyvinyl-pyrrolidone (PVP), sodium starch glycolate, microcrystalline cellulose, carboxymethylcellulose, and alginic acid are suitable disintegrating agents. Binding agents may include starch and gelatin. The lubricating agent, if present, may be magnesium stearate, stearic acid, sodium stearyl fumarate, or talc. If desired, the tablets may be coated with an esthetic coating based on, for example, hydroxypropyl methyl cellulose or polyvinyl alcohol copolymers together with wetting, anti-tacking, and/or coloring agents, and the like. If desired, the tablets may be coated with materials such as, for example, glyceryl monostearate or glyceryl distearate to delay absorption in the gastrointestinal tract, or may be coated with an enteric coating.

In addition to tablets, the single unit dosage forms of the disclosure may be presented as capsules, for example, hard and soft gelatin capsules. To prepare hard gelatin capsules, active agents of the disclosure of the disclosure may be mixed with a solid, semi-solid, or liquid diluent. Soft gelatin capsules may be prepared by mixing an active agent of the disclosure with water, an oil such as peanut oil or olive oil, liquid paraffin, a mixture of mono and di-glycerides of short chain fatty acids, polyethylene glycol 400, or propylene glycol.

Manufacturing a multi-component, single unit dosage form, in particular one that comprises four active agents, wherein at least one of those agents must be present in large amounts, for example, about 400 to about 1200 mg, preferably about 800 mg in the single unit dosage form, is challenging. One particular challenge is manufacturing a multi-component formulation that accommodates large dosage ranges and keeps the dosage form small enough for a subject to swallow. Single unit dosage forms of the disclosure can be prepared according to methods known in the art, for example, methods described in WO2013/004816 and WO2013004818, the entireties of which are incorporated by reference herein.

In preferred embodiments, the single unit dosage forms of the disclosure, when in the form of tablets, are of a sufficient structural integrity that they can be split, preferably into two or more pieces, for those subjects who have difficulties in swallowing larger-sized tablets. Score lines may be implemented to aid in splitting of the dosage forms.

The present disclosure is also directed to:
the protease inhibitor regimen as described above, comprising:
   darunavir, or a hydrate or solvate thereof;
   cobicistat;
   emtricitabine; and
   tenofovir alafenamide, or a pharmaceutically acceptable salt thereof; for use in a method of treating a human infected with an HIV virus and exhibiting an HIV viral load of less than or equal to 50 copies of HIV-1 virus particles per mL of blood plasma (<50 c/mL) by oral administration to the human, once daily,
wherein the human is treatment experienced and is switched to the protease inhibitor regimen from a first anti-retroviral regimen comprising:
   an integrase inhibitor,
   tenofovir alafenamide, or a pharmaceutically acceptable salt thereof, and
   emtricitabine;
and wherein the human has experienced a ≥10% increase in weight within 12 months' time period during the administration of the first anti-retroviral regimen; and
wherein the subject exhibits a viral load of less than or equal to 50 copies of HIV-1 virus particles per mL of blood plasma (50c/mL) after at least 24 weeks of the once-daily administration of the protease inhibitor regimen.

All disclosures and aspects of the features of the invention in relation to the method of treatment of a patient infected with an HIV virus, as set out above in this description also apply to the invention in relation to the same protease inhibitor regime, for use in a method of treatment of a patient infected with an HIV virus as described immediately above this paragraph and in appended claims.

The following examples are merely illustrative and are not intended to limit the disclosure to the materials, conditions, or process parameters set forth therein.

### EXAMPLES

Abbreviations

| | |
|---|---|
| AE | adverse event |
| AIDS | Acquired Immunodeficiency Syndrome |
| ALT | alanine aminotransferase |
| ARV | antiretroviral |
| ART | antiretroviral therapy |
| AST | aspartate aminotransferase |
| BMI | body metabolic index |
| BSQ-8D | body shape questionnaire |
| C or COBI | cobicistat |
| CI | confidence interval |
| cm | centimeters |
| D or DRV | darunavir |
| DAIDS | Division of Acquired Immunodeficiency Syndrome |
| DBP | diastolic blood pressure |
| D/C/F/TAF FDC | Darunavir/Cobicistat/Emtricitabine/Tenofovir |
| | Alafenamide Fixed-dose Combination |
| DEXA | dual-energy X-ray absorptiometry |
| eCrCl | estimated creatinine clearance |
| eGFRcr | creatinine-based estimated glomerular filtration rate |
| ESID | early study intervention discontinuation |
| F or FTC | emtricitabine |
| FDA | food drug administration |
| FDC | fixed-dose combination |
| FSH | follicle-stimulating hormone |
| FTC | emtricitabine |
| HAIR | hypertension, age, insulin, resistance |
| HBV | hepatitis B |
| HCV | hepatitis C virus |
| HIV | human immunodeficiency virus |
| HIV-1 RNA | human immunodeficiency virus type-1 |
| | ribonucleic acid |
| HIV-SI | HIV-Symptom Index |
| HOMA-IR | homeostatic model assessment of insulin resistance |
| ICF | informed consent form |
| INT | integrase |
| N | number |
| NAFLD | non-alcoholic fatty liver disease |
| NASH | nonalcoholic fatty liver disease, |
| PGIC | patient global impression of change |
| PGIC-S | patient global impression of change - Satiety |
| PI | protease inhibitor |
| PK | pharmacokinetic |
| PR | protease |
| PRO | patient reported outcomes |
| RAM | resistance-associated mutations |
| RT | reverse transcriptase |
| SAEs | serious adverse events |
| SBP | systolic blood pressure |
| TAF | tenofovir alafenamide |

Overall Design

This is a randomized, 48 week, active-controlled, open-label, prospective, multicenter, Phase 4 study of the safety and tolerability of switching to D/C/F/TAF FDC compared to continuing the current INI+TAF/FTC ARV regimen in virologically-suppressed HIV-1 infected adult subjects who have experienced rapid and significant body weight gain while receiving an INI+TAF/FTC ARV regimen.

Approximately 110 subjects will be included in this study. A maximum enrollment of approximately 70% male subjects will be utilized to ensure adequate recruitment of female subjects. A maximum enrollment of approximately 70% non-black subjects will be utilized to ensure adequate recruitment of diverse races. Eligible subjects are to:
have documented HIV-1 infection currently treated with a stable ARV regimen consisting of an INI combined with TAF/FTC for ≥6 consecutive months preceding the screening visit,
body mass index (BMI) of ≥18 kg/m² at the time of starting an INI+TAF/FTC ARV regimen,
have a rapid and significant weight gain, defined as a ≥10% increase in body weight within 12 months' time period while on the current INI+TAF/FTC ARV regimen,
be virologically-suppressed, with at least 1 plasma HIV-1 RNA measurement <50 copies/mL occurring between 12 and 2 months prior to screening while being on the stable INI+TAF/FTC ARV regimen and having HIV-1 RNA<50 copies/mL at the screening visit,
Not have had previous failure on DRV treatment or known documented history of ≥1 DRV resistance associated mutations (RAMs).

The study will consist of 3 phases:
Screening (approximately 30 days [up to a maximum of 6 weeks])
Open-Label Treatment (48 weeks)
Follow-up (for any subject who has an ongoing AE or serious adverse event (SAE) at the time of his/her last study visit).

Screening Phase

At baseline (Day 1), subjects who meet all eligibility criteria will be randomized in a 1:1 ratio to 1 of the following 2 treatment arms. Randomization will be stratified by sex (Male or Female) and race (Black/African American or Non-Black/African American) at Baseline. All enrolled subjects will be assigned randomized treatments in an open-label manner.
D/C/F/TAF FDC Arm (Immediate Switch): Switch to a regimen of DRV 800 mg+COBI 150 mg+FTC 200 mg+TAF 10 mg FDC once daily, (n=55) for 48 weeks;
Active-Control Arm (Delayed Switch): Continue current INI+TAF/FTC ARV regimen, (n=55) for 24 weeks. After 24 weeks subjects will switch to a regimen of DRV 800 mg+COBI 150 mg+FTC 200 mg+TAF 10 mg FDC once daily for an additional 24 weeks.

Treatment Phase

Subjects randomized to the Immediate Switch Arm must start D/C/F/TAF FDC within 24 hours of the Baseline visit and will continue to receive D/C/F/TAF treatment for a total of 48 weeks.

Subjects randomized to the Delayed Switch Arm, will continue their current INI+TAF/FTC ARV regimen for 24 weeks. After Week 24, all subjects in the Delayed Switch Arm will be given the option to receive the D/C/F/TAF FDC tablet and will be followed for an additional 24 weeks.

Subjects will return for study visits at Weeks 4, 12, 24, 36, and 48. Additionally, subjects randomized to the Delayed Switch Arm will have a study visit at Week 28 (i.e., 4 weeks after switching from the INI+TAF/FTC ARV regimen to D/C/F/TAF FDC).

Key metabolic assessments include body weight measurements, body composition assessed via dual-energy x-ray absorptiometry (DEXA) scan, waist circumference measurements, vital sign measurements, select clinical laboratory tests (including fasting lipids, fasting glucose, homeostatic model assessment of insulin resistance [HOMA-IR], HbA1c, leptin, adiponectin), and liver biomarkers. Key efficacy assessments include HIV-1 viral load, CD4⁺/CD8⁺ cell count, and HIV-1 genotype/phenotype resistance testing, if necessary. Key safety assessments will include AEs, physical examinations, standard clinical laboratory tests, and pregnancy testing. Concomitant medications will be recorded. D/C/F/TAF FDC study drug accountability and reasons for non-adherence will be monitored. Patient Reported outcomes (body shape questionnaire [BSQ-8D], DAILY EATS, HIV-Symptom Index [HIV-SI], and patient global impression of change [PGIC including PGIC-S]) will be completed.

Unscheduled visits can be conducted as needed based on individual tolerability issues, or virologic reasons (i.e., suspected virologic rebound) that occur between scheduled visits.

HIV-1 genotypic and phenotypic resistance testing will be performed for subjects (if any) with confirmed virologic rebound (2 consecutive HIV-1 RNA values ≥200 copies/mL at a scheduled or unscheduled visit) and a HIV-1 RNA >400 copies/mL at the time of confirmed rebound or at later timepoints. The confirmatory testing should be conducted 2 to 4 weeks after the initial HIV-1 RNA value ≥200 copies/mL. If genotypic/phenotypic resistance to study drug is determined, study drug may be discontinued, and the subject will be referred for continued medical care outside of the study if the decision is made to discontinue study drug.

Plasma concentrations of DRV and COBI may be determined in subjects (if any) experiencing virologic rebound using stored blood samples collected throughout the study, if deemed necessary. Plasma concentrations of INIs for the Delayed Switch Arm may be determined in subjects experiencing virologic rebound using stored blood samples collected throughout the study, if deemed necessary.

Screening for eligible subjects will be performed in a Screening Phase of approximately 30 days (up to maximum 6 weeks) starting from the signature of the ICF. During the Screening Visit (Day −30), subjects will undergo screening procedures outlined in Table 1. Subjects should not be randomized until all screening procedures have been completed and the subject meets the inclusion/exclusion criteria.

The inclusion and exclusion criteria for enrolling subjects in this study are described below.

Inclusion Criteria

| | |
|---|---|
| 1 | at least 18 years of age, inclusive. |
| 2 | BMI of ≥18 kg/m² at time of starting an INI + TAF/FTC |
| | ARV regimen. |
| 3 | documented HIV-1 infection. |
| 4 | currently being treated with a stable ARV regimen consisting of an |
| | INI combined with TAF/FTC for ≥6 consecutive months preceding |
| | the screening visit and experienced a ≥10% increase in body weight |
| | within a 12 months' time period while on the current INI TAF/FTC |
| | ARV regimen. |
| 5 | documented evidence of being virologically-suppressed while on the |
| | current stable INI + TAF/FTC ARV regimen (described above) prior |
| | to screening: at least 1 plasma HIV-1 RNA measurement <50 copies/ |
| | mL occurring between 12 and 2 months prior to the screening visit |
| | while on the stable INI + TAF/FTC ARV regimen and have HIV-1 |
| | RNA <50 copies/mL at the screening visit. |
| | A single viral load elevation of ≥50 copies/mL and <200 HIV-1 |
| | RNA copies/mL after previously reaching viral suppression within |
| | 12 months prior to screening is allowed, provided a subsequent viral |
| | load measurement is <50 HIV-1 RNA copies/mL prior to screening. |
| 6 | medically stable on the basis of physical examination, medical history, |
| | and vital signs, performed at screening. Any abnormalities, must be |
| | consistent with the underlying illness in the study population. |
| 7 | medically stable on the basis of clinical laboratory tests performed at |
| | screening. If the results of the serum chemistry panel including liver |
| | enzymes, other specific tests, blood coagulation, hematology, or |
| | urinalysis are outside the normal reference ranges, the subject may be |
| | included only if the abnormalities or deviations from normal are not |
| | clinically significant or to be appropriate and reasonable for the |
| | population under study. |
| 8 | must be able to swallow whole or split tablets. |
| 9 | Must have adequate insurance in place that will cover the current |
| | INI + TAF/FTC ARV regimen for at least 6 months. |
| 10 | must sign an ICF indicating that he or she understands the purpose of, |
| | and procedures required for, the study and is willing to participate in |
| | the study. |

Exclusion Criteria

| | |
|---|---|
| 1 | known history of malignancy within the past 5 years or ongoing |
| | malignancy other than cutaneous Kaposi's sarcoma, basal cell |
| | carcinoma, or resected, non invasive cutaneous squamous carcinoma. |
| 2 | known allergies, hypersensitivity, or intolerance to D/C/F/TAF FDC |
| | tablet or its excipients. |
| 3 | known active cryptococcal infection, active toxoplasmic encephalitis, |
| | Mycobacterium tuberculosis infection, or another AIDS-defining |
| | condition within 90 days prior to screening that would increase the |
| | risk of morbidity or mortality. |
| 4 | active hepatitis B (HBV) or hepatitis C virus (HCV) infection. |
| 5 | uncontrolled diabetes that will require treatment with insulin during |
| | the study period. |
| 6 | history of failure on DRV treatment orknown documented history of |
| | DRV RAMs. DRV RAMs include V11I, V32I, L33F, I47V, I50V, |
| | I54M/L, T74P, L76V, I84V, L89V (Note: If documented historical |
| | genotypes are available, the data must be made available to the |
| | sponsor for documentation of the criteria). If no historical genotype is |
| | available, the subject can be included, provided no previous failure on |
| | DRV treatment has been documented. |
| 7 | inadequate hematologic parameters at screening: platelets <50,000/μL, |
| | hemoglobin <8.5 g/dL, and absolute neutrophil count <1000/u/L. |
| 8 | evidence of Child Pugh Class C based on clinical laboratory |
| | testing and clinical evaluation. |
| 9 | screening hepatic transaminases (ALT and aspartate aminotransferase |
| | [AST]) >5x upper limit of the normal range (ULN). |
| 10 | screening creatinine based estimated glomerular filtration rate |
| | (eGFRcr) <30 mL/min according to the Cockcroft-Gault formula |
| | for creatinine clearance. |
| 11 | subjects initiating or discontinuing concomitant medications associated |
| | with significant changes in weight within the last 90 days: |
| | Diabetes Therapies: |
| | Insulin |
| | Thiazolidinediones; e.g.: pioglitazone. |
| | Sulfonylureas; e.g.: glipizide, glyburide, glimepiride, chlorpropamide, |
| | tolbutamide. |
| | Biguanides/Meglitinides; e.g.: Metformin, Nateglinide. |
| | Dipeptidyl peptidase-4 inhibitors; e.g.: linagliptin, saxagliptin, |
| | sitagliptin. |
| | Glucagon-like peptide-1 agonists; e.g.: exenatide, liraglutide, |
| | pramlintide. |
| | Alpha-Glucosidase inhibitors; e.g.: acarbose, miglitol. |
| | Psychiatric/Neurologic Therapies: |
| | Tricyclic antidepressants; e.g.: amitriptyline, doxepin, imipramine, |
| | nortriptyline, trimipramine. |
| | Selective Serotonin Reuptake Inhibitors; e.g.: sertraline, paroxetine, |
| | fluvoxamine. |
| | Antipsychotics; e.g.: haloperidol, loxapine, clozapine, chlorpromazine, |
| | fluphenazine, risperidone, olanzapine, quetiapine. |
| | Antiseizure/Anticonvulsants; i.e., Valproic acid, carbamazepine, |
| | gabapentin, topiramate, zonisamide, lamotrigine. |
| | Others: bupropion, nefazodone, lithium, mirtazapine. |
| | Steroid Hormones: chronic oral corticosteroids at an equivalent ≥5 mg |
| | of prednisoneorally a day |
| | Use of a methylprednisolone dose pack or use of acute steroids for |
| | treatment of allergic reactions for ≤7 days are not exclusionary. |
| | Hormone therapy/contraception; e.g.: estrogen, testosterone, |
| | progestogens, tesamorelin. |
| | Appetite Stimulants/Suppressants; e.g.: phentermine, topiramate, |
| | methylphenidate, amphetamine/dextroamphetamine, megestrol, |
| | oxandrolone, dronabinol. |
| 12 | receiving ongoing therapy with contraindicated drugs within 30 days |
| | of screening, not recommended drugs that cannot be adequately dose- |
| | adjusted, or subjects with any known allergies to the excipients of the |
| | D/C/F/TAF FDC tablet. |
| 13 | current alcohol or substance use to potentially interfere with subject |
| | study adherence or changes in body weight. |
| 14 | known, active, severe infections (other than HIV-1 infection) requiring |
| | parenteral antibiotic or antifungal therapy within 30 days prior to |
| | baseline. |
| 15 | received an investigational intervention (including investigational |
| | vaccines) or used an invasive investigational medical device within 90 |
| | days of baseline or is currently enrolled in an investigational study |
| | without prior approval from the sponsor. |
| 16 | pregnant, or breast-feeding, or planning to become pregnant while |
| | enrolled in this study or within 90 days after the last dose of study |
| | drug. |
| 17 | plans to father a child while enrolled in this study or within 90 days |
| | after the last dose of study drug. |
| 18 | any other condition or prior therapy for which participation would not |
| | be in the best interest of the subject (e.g., compromise the well-being) |
| | or that could prevent, limit, or confound the protocol-specified |
| | assessments. |
| 19 | unlikely to comply with the protocol requirements, |
| | based on clinical judgment. |

Administration

At the baseline visit (Day 1), subjects will be randomized in a 1:1 ratio to the Immediate Switch Arm (switch to D/C/F/TAF FDC) or the Delayed Switch Arm (maintain current INI+TAF/FTC ARV regimen). Randomization will be stratified by sex (Male or Female) and race (Black/African American or Non-Black/African American) at Baseline.
Immediate Switch Arm: D/C/F/TAF FDC (DRV 800 mg+COBI 150 mg+FTC 200 mg+TAF 10 mg) once daily for 48 weeks.
Delayed Switch Arm: continue current INI+TAF/FTC ARV regimen for 24 weeks. After 24 weeks subjects will switch to a regimen of D/C/F/TAF FDC (DRV 800 mg+COBI 150 mg+FTC 200 mg+TAF 10 mg) once daily for an additional 24 weeks.

Subjects randomized to the Immediate Switch Arm must start D/C/F/TAF FDC within 24 hours of the Baseline visit. D/C/F/TAF FDC tablet must be taken orally with food. subjects will be counseled to swallow D/C/F/TAF FDC tablet whole once daily at approximately the same time each day, according to their preference. D/C/F/TAF is to be taken with approximately 240 mL (8 ounces) of water. The D/C/F/TAF FDC tablet should be swallowed whole; alternatively, for subjects who are unable to swallow the tablet whole, D/C/F/TAF FDC may be split into 2 pieces using a tablet-cutter, and the entire dose should be consumed immediately after splitting. subjects should not attempt to dissolve the tablet in water.

If subjects notice that they have missed a D/C/F/TAF FDC intake and it is still within 12 hours of their regular dosing time, they should take the D/C/F/TAF FDC immediately with food. subjects can then continue their usual dosing schedule. If subjects notice that they have missed a dose more than 12 hours after the time it is usually taken, they should be instructed not to take it and simply resume the usual dosing schedule. subjects should not take a double dose to make up for a missed dose.

Each D/C/F/TAF FDC tablet contains darunavir ethanolate equivalent to 800 mg of darunavir, 150 mg of cobicistat, 200 mg of emtricitabine (FTC), and tenofovir alafenamide fumarate equivalent to 10 mg of tenofovir alafenamide (TAF). The yellow to yellowish-brown, capsule-shaped, filmcoated tablet is debossed with “8121” on one side and “JG” on the other side. D/C/F/TAF FDC is packaged in high-density polypropylene (HDPE) bottles with a silica gel desiccant and child-resistant closure.

For the Delayed Switch Arm, all components of the INI+TAF/FTC ARV regimen should be dosed and administered using the dosing schedule specified in the ARV agent's US Prescribing Information. Applicable procedures and treatment guidance based on the Prescribing Information should be respected.

Week 24 Objectives and Endpoints

| | |
|---|---|
| Objectives | Endpoints |

Primary

| | |
|---|---|
| To assess the percent change | Percent change from |
| in body weight when switching | Baseline in body weight |
| to D/C/F/TAF FDC (Immediate | at Week 24 |
| Switch Arm) compared to continuing | |
| the current INI + TAF/FTC ARV | |
| regimen (Delayed Switch Arm) | |
| in virologically-supressed (HIV-1 | |
| RNA <50 copies/mL) HIV-1 infected | |
| subjects who have experienced rapid | |
| and significant body weight gain | |

Secondary
Metabolic

| | |
|---|---|
| To assess changes in body weight | Change from Baseline in |
| when switching to D/C/F/TAF FDC | Absolute body weight |
| (Immediate Switch Arm) compared to | at Week 24 |
| continuing the current INI + TAF/FTC | Proportion of subjects with % |
| ARV regimen (Delayed Switch Arm) | change in body weight >5% |
| | at Week 24 |
| | Change from Baseline in |
| | BMI at Week 24 |
| To assess changes in body compostion | Change from Baseline in body |
| when switching to D/C/F/TAF FDC | composition as measured |
| (Immediate Switch Arm) compared to | be DEXA scan at Week 24 |
| continuing the current INI + TAF/FTC | Change from Baseline in |
| ARV regimen (Delayed Switch Arm) | waist circumference at Week 24 |
| To assess changes in blood pressure | Change in SBP and DBP |
| when switching to D/C/F/TAF FDC | from Baseline at Week 24 |
| (Immediate Switch Arm) compared to | |
| continuing the current INI + TAF/FTC | |
| ARV regimen (Delayed Switch Arm) | |
| To assess changes in clinical laboratory | Change from Baseline in |
| when switching to D/C/F/TAF FDC | fasting lipids at Week 24 |
| (Immediate Switch Arm) compared to | Change from Baseline in |
| continuing the current INI + TAF/FTC | fasting gluclose at Week 24 |
| ARV regimen (Delayed Switch Arm) | Change from Baseline in |
| | HOMA-IR at Week 24 |
| | Change from Baseline in |
| | HbA1c at Week 24 |
| | Change from Baseline in |
| | leptin and adiponectin at |
| | Week 24 |
| To assess changes in liver biomakers | Change from Baseline in |
| when switching to D/C/F/TAF FDC | the proportion of subjects |
| (Immediate Switch Arm) compared to | with advanced fibrosis |
| continuing the current INI + TAF/FTC | according to the NAFLD |
| ARV regimen (Delayed Switch Arm) | fibrosis score at Week 24 |
| | Change from Baseline in |
| | the proportion of subjects |
| | at high risk of NASH |
| | according to the HAIR |
| | score at Week 24 |
| To assess changes in concomitant | Proportion of subjects having |
| medications of interest (including | a dose-reduction or complete |
| anti-hyperextensive, anti-hyperglycemic, | withdrawal of anti-hypertensive, |
| and lipid lowering agents) when | hyperglycemic or lipid lowering |
| switching to D/C/F/TAF FDC | agents in the Immediate Switch |
| (Immediate Switch Arm) compared to | Arm or Delayed Switch Arm |
| continuing the current INI + TAF/FTC | from Baseline to Week 24 |
| ARV regimen (Delayed Switch Arm) | |

Safety

| | |
|---|---|
| To evaluate the safety of switching to | Incidence of any Grade AEs |
| D/C/F/TAF FDC (Immediate Switch | (related and not related) |
| Arm) compared to continuing | through Week 24 |
| the current INI + TAF/FTC | Incidence of any Grade 3 and |
| ARV regimen (Delayed Switch Arm) | 4 AEs (related and not related) |
| | through Week 24 |
| | Incidence of discontinuations |
| | due to AEs through Week 24 |
| | Incidence of SAEs (related and |
| | not related) through Week 24 |
| | Change from Baseline in clinical |
| | laboratory tests through Week 24 |
| | Incidence of Grade 3 and 4 |
| | laboratory abnormalities |
| | through Week 24 |

Efficacy

| | |
|---|---|
| To evaluate the virologic outcomes | Proportion of subjects with |
| when switching to D/C/F/TAF FDC | confirmed virologic rebound |
| (Immediate Switch Arm) compared to | through Week 24 |
| continuing the current INI + TAF/FTC | Proportion of subjects with |
| ARV regimen (Delayed Switch Arm) | virologic response (HIV-1 |
| | RNA <50 copies/mL) at |
| | Week 24 according to the |
| | FDA snapshot algorithim |
| | Proportion of subjects with |
| | virologic failure (HIV-1 |
| | RNA ≥50 copies/mL) at |
| | Week 24 according to |
| | the FDA snapshot algorithim |
| | Proportion of subjects having |
| | virologic response (HIV-1 |
| | RNA <200 copies/mL) at |
| | Week 24 according to the |
| | FDA snapshot algorithim |
| | Proportion of subjects having |
| | virologic failure (HIV-1 |
| | RNA ≥200 copies/mL) at |
| | Week 24 according to the |
| | FDA snapshot algorithim |
| To evaluate immunological changes | Change from Baseline in |
| when switcing to D/C/F/TAF FDC | CD4+ cell count 1 in Week 24 |
| (Immediate Switch Arm) compared to | |
| continuing the current INI + TAF/FTC | |
| ARV regimen (Delayed Switch Arm) | |

Resistance

| | |
|---|---|
| To asses viral resistance in subjects | Proportion of subjects with , |
| with confirmed HIV-1 RNA rebound for | pre-baseline PR, RT, and INI |
| subjects switching to D/C/F/TAF FDC | resistance-associated mutations |
| (Immediate Switch Arm) compared to | (RAMs) based on historical |
| continuing the current INI + TAF/FTC | genotypes |
| ARV regimen (Delayed Switch Arm) | Incidence of observed genotypic |
| | and phenotypic ARV resistance |
| | for subjects meeting HIV-1 RNA |
| | rebound criteria through Week 24 |
| | Proportions of subject with newly |
| | identified post-baseline RAMs |
| | and phenotypic resistance, compared |
| | to pre-baseline resistance tests when |
| | available, upon meeting confirmed |
| | virologic rebound through Week 24 |

PROs

| | |
|---|---|
| To assess changes in the burden of | Change from Baseline in |
| common symptons associated with | the proportion of subjects who |
| HIV treatment of disease for subjects | have bothersome symptoms |
| switching to D/C/F/TAF FDC | (scores of 2, 3, or 4) across |
| (Immediate Switch Arm) compared to | all items of the HIV-SI at |
| continuing the current INI + TAF/FTC | Week 24 |
| ARV regimen (Delayed Switch Arm) | Change from Baseline in the |
| | proportion of subjects who |
| | have any symptoms (scores of 1, |
| | 2, 3, or 4) across all items of the |
| | HIV-SI at Week 24 |
| | Association between treatment |
| | arm and each bothersome symptom |
| | of the HIV-SI adjusting for |
| | Baseline variables at Week 24 |
| To describe responses on the PGIC in | PGIC at Week 24 |
| subjects switching to D/C/F/TAF FDC | |
| (Immediate Switch Arm) and in subjects | |
| continuing the current INI + TAF/FTC | |
| ARV regimen (Delayed Switch Arm) | |

Adherence

| | |
|---|---|
| To evaluate adherence in subjects | Adherence rates by subject |
| switching to D/C/F/TAF FDC | self-report using 4-day recall |
| (Immediate Switch Arm) and in | at Week 4, 12, and 24 |
| subjects continuing the current INI + | |
| TAF/FTC ARV regimen | |
| (Delayed Switch Arm) | |

Exploratory
Exploratory clinical biomarker

| | |
|---|---|
| To assess changes in alpha melancyte- | Change from Baseline in alpha |
| simulating hormone when switching | melanocyte-stimulating hormone |
| to D/C/F/TAF FDC | at Week 24 |
| (Immediate Switch Arm) compared to | |
| continuing the current INI + TAF/ | |
| FTC ARV regimen | |
| (Delayed Switch Arm) | |

Exploratory PROs

| | |
|---|---|
| To assess changes in eating-related | Change from Baseline in |
| concepts (hunger, appetite, cravings, | the scores on the DAILY |
| and safety) for subjects switching to | EATS at Week 24 |
| D/C/F/TAF FDC (Immediate Switch | |
| Arm) compared to continuing the | |
| current INI + TAF/FTC ARV regimen | |
| (Delayed Switch Arm) | |
| To assess changes in concerns about | Change from Baseline |
| body shape for subjects switching to | in the scores on the |
| D/C/F/TAF FDC (Immediate Switch | BSQ-8D at Week 24 |
| Arm) compared to continuing the | Change from Baseline in the |
| current INI + TAF/FTC ARV regimen | proportion of subjects who |
| (Delayed Switch Arm) | have no concern (<19), |
| | mild concern (19-25), |
| | moderate concern (26-33) |
| | or marked concern (>33) |
| | with their body shape at |
| | Week 24 |

Secondary objectives/endpoints at Week 48 are similar to the Week 24 Objectives and Endpoints and thus will be used to assess 1) outcomes for the Immediate Switch Arm at Week 48 and 2) the consistency of effect on outcomes from Baseline to Week 24 in the Immediate Switch Arm versus the Week 24 to Week 48 in the Delayed Switch Arm.

Assessments and Procedures

Percent Change in Body Weight

Body weight will be evaluated at specified time points. Body weight will be measured using a calibrated scale. Attention should be given to weigh the subject on the same scale through the duration of the study. Subjects should be weighed wearing underwear and a gown. Subjects will be instructed to take off their shoes and to empty their bladders before being weighed. If disrobing for weighing is logistically impossible, the subject must be dressed as lightly as possible, with consistency from visit to visit. The scale should be calibrated according to the manufacturers specifications and at the frequency recommended by the manufacturer before the first subject is weighted. Calibration must be documented in the calibration log.

Metabolic Assessments

Systolic and diastolic blood pressure (SBP, DBP), pulse rate (supine after at least 5 minutes rest) will be recorded in a quiet setting without distractions, at specified time points. Blood pressure and pulse/heart rate measurements will be assessed with a completely automated technique. Manual techniques will be used only if an automated device is not available. Body Mass Index will be calculated using body weight and height measured at screening. Height will be measured using a wall-mounted stadiometer or one mounted on a balance beam scale, whichever is most appropriate for the individual subject. Subjects should be wearing socks or barefoot and should not be wearing shoes.

Body composition will be assessed at specified time points via whole body DEXA scans. DEXA scans may be performed between screening and baseline (+2 weeks), at Weeks 24, 48, and the ESID visit (±10 days) (only to be performed at ESID if the last scan is more than 12 weeks from the date of the ESID visit and the ESID visit takes place before Week 48). A rescan for technical reasons at all scheduled time points is allowed within 2 weeks. A complete description of the procedures for the DEXA scans will be provided in the DEXA manual. Reading of the DEXA scans will be performed centrally. Outputs from the DEXA scans will be used to measure fat composition in the subjects. Specific regions of interest will include trunk, Visceral Adipose Tissue (VAT), Total Body, Adjusted Total Body (Total Body minus Head), and Appendages (Legs and Arms). For each region of interest, absolute mass of fat, lean body mass and total mass will be reported. The percent of fat relative to each region will also be reported. Visceral adipose tissue will be reported in mass and volume.

Waist circumference will be measured at specified time points. Waist circumference will be measured with the subject standing, wearing underwear, with or without a gown. The measurement will be performed at a level midway between the superior aspect of the iliac crests and the lower lateral margin of the ribs. The measurement need not be at the level of the umbilicus. The measuring tape will be kept horizontal. Non-alcoholic fatty liver disease (NAFLD) Fibrosis and hypertension, age, insulin, resistance (HAIR) scores will be calculated based on receipt of central laboratory assessments and clinical status of the subject.

Fasting clinical laboratory tests will be assessed at specified time points to assess changes in the following metabolic parameters: lipids, calculated homeostatic model assessment of insulin resistance (HOMA-IR), HbA1C, leptin, adiponectin, and alpha melanocyte-stimulating hormone.

Antiviral Efficacy and Immunologic Change

Blood samples for determination of plasma HIV-1 RNA viral load and immunologic parameters will be taken at specified time points.

Plasma viral load will be measured using a validated assay at a central laboratory (i.e., Roche COBAS AmpliPrep/COBAS TaqMan HIV-1 Test, Version 2.0.) The assay linear range is 20 to 10,000,000 copies/mL with a lower limit of quantification (LLOQ) of 20 copies/mL and a limit of detection (LOD) of 20 copies/mL). Immunologic change will be determined by changes in CD4⁺ cell count (absolute and %).

Changes in viral load, changes in CD4⁺ cell counts (either decreases or increases) or detected resistance will be part of the efficacy analysis and should not be reported as AEs or SAEs.

Resistance Determinations

Samples for HIV-1 genotype/phenotype resistance testing will be taken at the specified time points.

For subjects (if any) with confirmed virologic rebound (2 consecutive HIV-1 RNA values ≥200 copies/mL at a scheduled or unscheduled visit) and with a HIV-1 RNA value ≥400 copies/mL, HIV-1 genotypic/phenotypic resistance testing will be performed on the confirmed rebound sample if HIV-1 RNA ≥400 copies/mL or on a following visit with HIV-1 RNA ≥400 copies/mL. Other time points may still be analyzed if deemed necessary by the sponsor.

Safety Assessments

Clinical events and clinically significant laboratory abnormalities will be graded according to the Division of AIDS (DAIDS).

Physical Examination

A complete physical examination will be conducted. Complete physical examinations include general appearance; skin (and mucus membranes); eyes; ears, nose, and throat; head, neck, and thyroid; heart; lung, chest (incl. breasts); abdomen; genitalia; anorectal; lymph nodes; musculoskeletal; and neurological. Urogenital/anorectal examination will be performed if clinically relevant. Subjects should be undressed during the complete physical examinations, which should be performed by a licensed medical doctor, physician's assistant or nurse practitioner in accordance with local guidelines. Physical examination will also include evidence of ascites or encephalopathy at Screening in order to fully calculate Child Pugh Class.

Vital Signs

Pulse/heart rate (supine after at least 5 minutes rest) and blood pressure will be assessed will be conducted at all study visits.

Blood pressure and pulse/heart rate measurements should be preceded by at least 5 minutes of rest in a quiet setting without distractions (e.g., television, cell phones).

Clinical Safety Laboratory Assessments

Blood samples for serum chemistry and hematology and a urine sample for urinalysis will be collected.

Patient-Reported Outcomes

Following PROs will be assessed. Subjects will complete the PROs using electronic devices. The BSQ-8D, HIV-SI and PGIC (including PGIC-S) should be completed by the subjects during the study visit before all other study-related procedures to prevent influencing subject perceptions. The PGIC and PGIC-S should be completed after the subject completes the BSQ-8D and HIV-SI.

HIV-Symptom Index (HIV-SI)

The HIV-SI is a validated PRO instrument that assesses the burden of 20 common symptoms associated with HIV treatment or disease. Respondents are asked about their experience with each of 20 symptoms during the past 4 weeks using a 5-point, Likert-type scale. Response options and scores are as follows: (0) “I don't have this symptom;” (1) “I have this symptom and it doesn't bother me;” (2) “I have this symptom and it bothers me a little;” (3) “I have this symptom and it bothers me;” (4) “I have this symptom and it bothers me a lot.” The 20 symptoms comprising the HIV-SI are fatigue/loss of energy, difficulty sleeping, nervous/anxious, diarrhea/loose bowels, changes in body composition, feeling sad/down/depressed, bloating/pain/gas in stomach, muscle aches/joint pain, problems with sex, trouble remembering, headaches, pain/numbness/tingling in hands/feet, skin problems/rash/itching, cough/trouble breathing, fever/chills/sweats, dizzy/lightheadedness, body weight loss/wasting, nausea/vomiting, hair loss/changes, and loss of appetite/food taste. Symptoms scores can be dichotomized into not bothersome (scores of 0 or 1) or bothersome (scores of 2, 3 and 4) and overall bothersome symptom count at baseline can be generated by counting the number of individual symptoms scored as bothersome.

Body Shape Questionnaire (BSQ-8D)

The BSQ-8D is an 8-item version of the 34-item self-report questionnaire that was developed and validated to measure concerns about body shape; in particular, it focused on the phenomenal experience of “feeling fat”. Respondents are asked about how they have been feeling about their appearances over the past four weeks. Each item from this questionnaire is answered using a 6-point Likert scale: 1 (never), 2 (rarely), 3 (sometimes), 4 (often), 5 (very often), and 6 (always) and the overall score is the total across the 8 items.

Daily Eats

The DAILY EATS will be administered to measure eating-related concepts such as hunger, appetite, cravings, and satiety. The home-based DAILY EATS should be completed daily, preferably in the evening, and, whenever possible, in the same setting for 7 consecutive days. The site should contact subjects approximately 7 days prior to the Baseline (Day 1), Week 24 and Week 48 visits, preferably by telephone, to remind the completion of the DAILY EATS for 7 consecutive days prior to the applicable visit. For subjects who discontinue early from study drug, no DAILY EATS completion is required at the ESID visit.

Patient Global Impression of Change

The PGIC is a global index that is used to rate the overall status of the subject related to the subject's overall condition. It is rated by the subject and is based on the single question, “Compared to before starting the study or compared to the Week 24 visit, my overall status is,” where response choices include 1=very much improved, 2=much improved, 3=minimally improved, 4=no change, 5=minimally worse, 6=much worse, and 7=very much worse.

The single-item PGIC for satiety (PGIC-S), with question, “Compared to before starting the study or compared to the Week 24 visit, how would you rate your satisfaction (fullness) after meals in the past 7 days?” and where response choices include 1=much more satisfied (full), 2=moderately more satisfied (full), 3=a little more satisfied (full), 4=no change, 5=a little less satisfied (full), 6=moderately less satisfied (full), and 7=much less satisfied (full), will also be used. The content validity of the PGIC-S has been demonstrated in overweight and obese patients with and without T2DM, although the psychometric properties of the PGIC-S were not evaluated.

Renal

eCrCl will be calculated according to the Cockcroft-Gault formula and will be followed post baseline during the treatment phase.

Alpha Melanocyte-Stimulating Hormone

Levels of alpha melanocyte-stimulating hormone will be collected.

Leptin

Leptin levels will be collected.

Adiponectin

Adiponectin levels will be collected.

What is claimed is:

1. A method of treating a human infected with an HIV virus and exhibiting an HIV viral load of less than or equal to 50 copies of HIV-1 virus particles per mL of blood plasma (<50 c/mL), comprising:
orally administering to the human, once daily, a protease inhibitor regimen comprising:
darunavir, or a hydrate or solvate thereof;
cobicistat;
emtricitabine; and
tenofovir alafenamide, or a pharmaceutically acceptable salt thereof;
wherein the human is treatment experienced and is switched to the protease inhibitor regimen from a first anti-retroviral regimen comprising:
an integrase inhibitor,
tenofovir alafenamide, or a pharmaceutically acceptable salt thereof, and
emtricitabine;
and wherein the human has experienced a ≥10% increase in weight within a 12-month time period during the administration of the first anti-retroviral regimen; and
wherein the subject exhibits a viral load of less than or equal to 50 copies of HIV-1 virus particles per mL of blood plasma (≤50 c/mL) after at least 24 weeks of the once-daily administration of the protease inhibitor regimen.

2. The method of claim 1, wherein the integrase inhibitor is bictegravir, dolutegravir, elvitegravir, elvitegravir/cobicistat, or raltegravir.

3. The method of claim 1, wherein the protease inhibitor regimen comprises:
darunavir,
cobicistat,
emtricitabine, and
tenofovir alafenamide or a pharmaceutically acceptable salt thereof.

4. The method of claim 1, wherein the protease inhibitor regimen comprises:
a darunavir hydrate,
cobicistat,
emtricitabine, and
tenofovir alafenamide or a pharmaceutically acceptable salt thereof.

5. The method of claim 1, wherein the protease inhibitor regimen comprises:
a darunavir solvate,
cobicistat,
emtricitabine, and
tenofovir alafenamide or a pharmaceutically acceptable salt thereof.

6. The method of claim 1, wherein the protease inhibitor regimen comprises:
darunavir ethanolate,
cobicistat,
emtricitabine, and
tenofovir alafenamide or a pharmaceutically acceptable salt thereof.

7. The method of claim 1, wherein the protease inhibitor regimen comprises:
darunavir ethanolate,
cobicistat,
emtricitabine, and
tenofovir alafenamide fumarate.

8. The method of claim 1, wherein the protease inhibitor regimen comprises:
about 800 mg of darunavir,
about 150 mg of cobicistat,
about 200 mg of emtricitabine, and
about 10 mg of tenofovir alafenamide.

9. The method of claim 1, wherein the protease inhibitor regimen is provided as a single unit dosage form.

10. The method of claim 1, wherein the human does not exhibit a clinically significant change in body weight, as compared to the body weight of the human prior to administering the protease inhibitor regimen, after about 24 or about 48 weeks.

11. The method of claim 1, wherein the human exhibits a clinically significant change in body weight, as compared to the body weight of the human prior to administering the protease inhibitor regimen, after about 24 or about 48 weeks.

12. The method of claim 1, wherein the human has a >5% weight loss, as compared to the weight of the human prior to administering the protease inhibitor regimen, after about 24 or about 48 weeks.

13. The method of claim 1, wherein the human exhibits a clinically significant improvement in body composition as measured by dual-energy X-ray absorptiometry (DEXA), as compared to the body composition as measured by DEXA of the human prior to administering the protease inhibitor regimen, after about 24 or about 48 weeks.

14. The method of claim 1, wherein the human exhibits no clinically significant change in body composition as measured by dual-energy X-ray absorptiometry (DEXA), as compared to the body composition as measured by DEXA of the human prior to administering the protease inhibitor regimen, after about 24 or about 48 weeks.

15. The method of claim 1, wherein the human exhibits a clinically significant improvement in waist circumference, as compared to the waist circumference of the human prior to administering the protease inhibitor regimen, after about 24 or about 48 weeks.

16. The method of claim 1, wherein the human exhibits no clinically significant change in waist circumference, as compared to the waist circumference of the human prior to administering the protease inhibitor regimen, after about 24 or about 48 weeks.

17. The method of claim 1, wherein the human exhibits a clinically significant improvement in at least one of systolic blood pressure and diastolic blood pressure, as compared to the blood pressure of the human prior to administering the protease inhibitor regimen, after about 24 or about 48 weeks.

18. The method of claim 1, wherein the human exhibits no clinically significant change in at least one of systolic blood pressure and diastolic blood pressure, as compared to the blood pressure of the human prior to administering the protease inhibitor regimen, after about 24 or 48 weeks.

19. The method of claim 1, wherein the human exhibits a clinically significant improvement in fasting lipids, as compared to the fasting lipids of the human prior to administering the protease inhibitor regimen, after about 24 or about 48 weeks.

20. The method of claim 1, wherein the human exhibits no clinically significant change in fasting lipids, as compared to the fasting lipids of the human prior to administering the protease inhibitor regimen, after about 24 or about 48 weeks.

21. The method of claim 1, wherein the human exhibits a clinically significant improvement in fasting glucose, as compared to the fasting glucose of the human prior to administering the protease inhibitor regimen, after about 24 or about 48 weeks.

22. The method of claim 1, wherein the human exhibits no clinically significant change in fasting glucose, as compared to the fasting glucose of the human prior to administering the protease inhibitor regimen, after about 24 or about 48 weeks.

23. The method of claim 1, wherein the human has a clinically significant decrease in homeostatic model assessment of insulin resistance (HOMA-IR), as compared to the HOMA-IR of the human prior to administering the protease inhibitor regimen, at about 24 or about 48 weeks.

24. The method of claim 1, wherein the human has no clinically significant change in homeostatic model assessment of insulin resistance (HOMA-IR), as compared to the HOMA-IR of the human prior to administering the protease inhibitor regimen, at about 24 or about 48 weeks.

25. The method of claim 1, wherein the human exhibits a clinically significant improvement in HbA1c, as compared to the HbA1c of the human prior to administering the protease inhibitor regimen, after about 24 or about 48 weeks.

26. The method of claim 1, wherein the human exhibits no clinically significant change in HbA1c, as compared to the HbA1c of the human prior to administering the protease inhibitor regimen, after about 24 or about 48 weeks.

27. The method of claim 1, wherein the HIV infection is an HIV-1 infection.

28. The method of claim 1, wherein the human is 18 years of age or older.

29. The method of claim 1, wherein the human is less than 18 years of age.

30. The method of claim 1, wherein the human weighs at least 40 kg.
